(19) United States
(12) Patent Application Publication
(10) Pub. No.: US 2009/0198311 A1
(43) Pub. Date: Aug. 6, 2009

(54) COOLING BLANKET

(76) Inventors: Cherlin Johnson, Los Angeles, CA (US); Aimee Moulin, Culver City, CA (US)

Correspondence Address: DAVIS WRIGHT TREMAINE LLP/Los Angeles, 865 FIGUEROA STREET, SUITE 2400, LOS ANGELES, CA, 90017-2566, US

(21) Appl. No.: 12/418,837

(22) Filed: Apr. 6, 2009

Related U.S. Application Data
(63) Continuation of application No. 11/317,476, filed on Dec. 22, 2005.

(51) Int. Cl. A61F 7/00

(52) U.S. Cl. 607/109; 607/114

(57) ABSTRACT

The invention provides a cooling blanket useful for lowering the body temperature of a mammal, such as a human. The cooling blanket may be particularly useful when used in connection with a deleterious cardiac event, such as myocardial infarction. The cooling blanket is activated when reagents, such as ammonium nitrate, urea, and/or water, are placed in contact with one another; thereby producing an endothermic reaction.

### CROSS-REFERENCE TO RELATED APPLICATIONS

This application makes a claim of priority as a continuation of U.S. application Ser. No. 11/317,476, filed Dec. 22, 2005, now pending.

### FIELD OF ART

This invention relates to the field of medical equipment. Particularly, the invention provides for a cooling blanket that is useful for lowering the body temperature of a mammal.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

It has been shown that there are instances where it may be clinically beneficial to lower the body temperature of a human or other mammal. See, e.g., S. Bernard et al., Treatment of comatose survivors of out-of-hospital cardiac arrest with induced hypothermia, N. Engl. J. Med., 346(8):557-563 (2002); D. Marion et al., Treatment of traumatic brain injury with moderate hypothermia, N. Engl. J. Med., 346(8):557-563 (2002). For instance, during or after an unfavorable cardiac event, such as acute myocardial infarction or “heart attack,” lowering a patient's body temperature may result in a reduction in damage to the heart and other organs. While this may be due to a variety of physiological processes, it is generally believed that cooling the body slows down certain bodily functions and therefore decreases the potential damage that a cardiac incident such as this may have on the heart and other organs. There are other instances, too, where cooling is generally believed to be beneficial. This includes, by way of example, in instances of head trauma, stroke, heat stroke, hyperthermia (and, generally, in temperature maintenance), fever, seizure, neonatal asphyxiation, drowning, respiratory failure, and sepsis. See, e.g., Z-J Zheng et al., Sudden cardiac death in the United States, 1989 to 1998, Circ., 104:2158-2163 (2001); I. G. Jacobs et al., The chain of survival, Ann. Emerg. Med., 37:S5-S16 (2001); J. C. Finn et al., Outcomes of out-of-hospital cardiac arrest patients in Perth, Western Australia, Resuscitation, 51:247-255 (2001); S. A. Bernard, Outcome from prehospital cardiac arrest in Melbourne, Australia, Emerg. Med. 10:25-29 (1998); K. L. Smith et al., Cardiac arrests treated by ambulance paramedics and fire fighters, Med. J. Aust., 177:305-309 (2002); The Public Access Defibrillation Trial Investigators, Public-access defibrillation and survival after out-of-hospital cardiac arrest, N. Engl. J. Med., 351:637-646 (2004); I. G. Stiell et al., Advanced cardiac life support in out-of-hospital cardiac arrest, N. Engl. J. Med., 351:647-656 (2004); C. M. Booth et al., Is this patient dead, vegetative, or severely neurologically impaired? Assessing outcome for comatose survivors of cardiac arrest, JAMA, 291:870-879 (2004); J. P. Williamson et al., Near-drowning treated with therapeutic hypothermia, Med. J. Aust., 181:500-501 (2004); S. Bernard et al., Induced hypothermia in critical care medicine: A review, Crit. Care Med., 31:2041-2051 (2003); Hypothermia After Cardiac Arrest Study Group, Mild therapeutic hypothermia to improve the neurological outcome after cardiac arrest, N. Engl. J. Med., 346:549-556 (2002); J. Nolan et al., Therapeutic hypothermia after cardiac arrest. An advisory statement by the Advanced Life Support Task Force of the International Liaison Committee on Resuscitation, Resuscitation. 57:231-235 (2003); A. Meyers et al., Asystolic cardiac arrest in Melbourne, Australia, Emerg. Med., 13:186-189 (2001); S. Bernard et al., Induced hypothermia using large volume, ice-cold intravenous fluid in comatose survivors of out-of-hospital cardiac arrest: a preliminary report, Resuscitation, 56:9-13 (2003); P. Safar et al., Therapeutic hypothermia after cardiac arrest, N. Engl. J. Med., 346(8):612-613 (2002); P. Safar et al., Cerebral resuscitation potentials for cardiac arrest, Crit. Care Med., 30(4 Supp):S140-144 (2002); R. Hickey et al., Delayed, spontaneous hypothermia reduces neuronal damage after asphyxial cardiac arrest in rats, Crit. Care Med., 28(10):3511-3516 (2000); D. Marion, Therapeutic moderate hypothermia and fever, Curr. Pharma. Des., 7:1533-1536 (2001); M. Thoresen et al., Cardiovascular changes during mild therapeutic hypothermia and rewarming in infants with hypoxic-ischemic encephalopathy, Pediatrics, 106(1) 92-99 (2000); and D. Giorgiadis et al., Endovascular cooling for moderate hypothermia in patients with acute stroke, Stroke, 32:2550-2553 (2001).

Despite this belief in the medical community, there are few medical devices that can be used to conveniently effect body cooling commensurate with the aforementioned perceived benefits. Individual cold packs available from a number of commercial sources in an array of configurations may be used for this purpose, but without a mechanism to interconnect such packs in a fast and predictable fashion, it is at least largely inconvenient to provide consistent cooling across the body surface. This is especially problematic in instances where it is desirable to provide this form of cooling to a patient under conditions that do not readily facilitate the balancing and careful placement of a series of cooling packs. By way of example, this technique is especially desirable in the emergency medical setting, such as in an ambulance and/or when a patient is being moved from the location where he first suffered a deleterious cardiac event (e.g., at home, at work, in the car, etc.) to a location where more expansive care can be offered (e.g., at a hospital or clinic). Balancing and/or carefully placing numerous cold packs would be impractical under such circumstances, and it may be especially challenging to maintain a series of cold packs underneath a patient that is lying on his back, such as on a gurney or stretcher, or even on a bed.

There thus exists a need in the art for convenient and transportable equipment for lowering a mammal's body temperature, as the aforementioned challenges and potential benefits apply equally to humans as well as other animals (i.e., in the veterinary setting, with both large and small animals).

### SUMMARY OF THE INVENTION

The following embodiments of the invention and aspects thereof are described and illustrated in conjunction with compositions and methods which are meant to be exemplary and illustrative, not limiting in scope.

In one embodiment, the invention includes a cooling blanket to cool a mammal, comprising a top layer, a bottom layer, and at least one compartment formed between the top layer and the bottom layer, the at least one compartment containing a first reagent. The first reagent may be selected from the group consisting of ammonium nitrate, urea, water, and combinations thereof. The top layer and the bottom layer may each include at least one sub-layer. The cooling blanket may further include a thermometer in communication with the at least one compartment to indicate if the compartment has been activated. The at least one compartment may contain a cold pack, and may include an external closure to permit the removable introduction of the cold pack. The cooling blanket may include a bladder to hold a second reagent, wherein the at least one compartment is in fluid communication with an internal portion of the cooling blanket adjacent to the bladder, the bladder is adapted to rupture upon the application of a predetermined amount of external force, and the first reagent and the second reagent are adapted to produce an endothermic reaction upon mixing. The at least one compartment may include a seal that separates the first reagent from a second reagent, wherein the seal is adapted to rupture upon the application of a predetermined amount of external force on the compartment, and wherein the first reagent and the second reagent are adapted to produce an endothermic reaction upon mixing. The at least one compartment may include a sack containing a second reagent, wherein the first reagent is contained outside the sack, wherein the sack is adapted to rupture upon the application of a predetermined amount of external force on the compartment, and wherein the first reagent and the second reagent are adapted to produce an endothermic reaction upon mixing. The cooling blanket may further include an inlet valve adapted to permit the introduction of a second reagent to the cooling blanket, wherein the first reagent and second reagent are adapted to produce an endothermic reaction upon mixing, and wherein each of the at least one compartment is linked to at least one other compartment by a one-way valve, whereby introduction of the second reagent through the inlet valve causes a mixing of the first reagent with the second reagent in each of the at least one compartment. The cooling blanket may further include at least one opening adapted to allow a limb or appendage of a mammal to extend through the cooling blanket. The cooling blanket may further include a hood to cover a head of a mammal. The cooling blanket may further include a fastening apparatus adapted to fasten the cooling blanket to another cooling blanket. The fastening apparatus may be selected from the group consisting of a grommet system, a tie system, a button system, a hook and loop system, a zipper system, and combinations thereof. The cooling blanket may further include a securing apparatus adapted to secure the cooling blanket to the mammal at its neck, limb, or appendage. The securing apparatus may be selected from the group consisting of a grommet system, a tie system, a button system, a hook and loop system, a zipper system, and combinations thereof. The cooling blanket may further include a stabilizing apparatus adapted to stabilize the cooling blanket with respect to the mammal. The cooling blanket may further include an instruction for cardiac life support.

In a second embodiment, the invention includes a case for containing a cooling blanket to cool a mammal, comprising a hollow unit, a top removably attached to the hollow unit, and a bottom removably attached to the hollow unit, wherein the cooling blanket comprises a top layer, a bottom layer, and at least one compartment formed between the top layer and the bottom layer, the at least one compartment containing a first reagent and a second reagent that are adapted to produce an endothermic reaction upon mixing. The case may further include at least one strap adapted to be wrapped around the case to squeeze and activate the cooling blanket.

In a third embodiment, the invention includes a kit, comprising a cooling blanket to cool a mammal, and instructions for activating the cooling blanket to cool a mammal, wherein the cooling blanket comprises a top layer, a bottom layer, and at least one compartment formed between the top layer and the bottom layer, the at least one compartment containing a first reagent and a second reagent that are adapted to produce an endothermic reaction upon mixing. The kit may further include an additional component selected from the group consisting of a cold pack, a container of water, a water pumping device, and combinations thereof. The kit may further include a case to contain the cooling blanket.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts a cooling blanket, in accordance with an embodiment of the present invention. The cooling blanket includes a series of compartments; one of which is depicted with an opening with an external closure to permit the removable introduction of a cold pack.

FIG. 2 depicts a cooling blanket in cross-section, in accordance with an embodiment of the present invention. A compartment of the cooling blanket contains a cold pack.

FIG. 3 depicts a cooling blanket with a network of compartments interconnected by a series of one-way valves, in accordance with an embodiment of the present invention. The cooling blanket also includes an inlet valve.

FIG. 4 depicts a cooling blanket with a series of compartments, each including a seal that separates two reagents from one another, in accordance with an embodiment of the present invention.

FIG. 5 depicts a cooling blanket with a series of compartments, each including a sack containing a reagent, in accordance with an embodiment of the present invention. The sack is depicted as either freely floating inside the compartment, or anchored to an internal portion of a compartment wall.

FIG. 6 depicts a cooling blanket with a bladder and a series of compartments in fluid communication with the internal portion of the blanket adjacent to the bladder, in accordance with an embodiment of the present invention. FIG. 6A depicts a schematic view of the cooling blanket. FIG. 6B depicts the cooling blanket in cross-section along line A-A.

FIG. 7 depicts a cooling blanket with openings to accommodate the arms and head and neck of a mammal, in accordance with an embodiment of the present invention. FIGS. 7A, 7B, and 7C depict the front, back, and side perspective views of a cooling blanket with openings to accommodate the arms and head and neck of a mammal. FIGS. 7D and 7E depict the front and side perspective views of a cooling blanket with openings to accommodate the arms and head and neck of a mammal, but also including a hood to cover the mammal's head.

FIG. 8 depicts a unitary cooling blanket that may be wrapped around a mammal, in accordance with an embodiment of the present invention.

FIG. 9 depicts a cooling blanket system including multiple cooling blankets mechanically secured to one another, in accordance with an embodiment of the present invention. A fastening apparatus (tie system) that may be used to fasten the cooling blankets to one another is illustrated.

FIG. 10 depicts a cooling blanket with securing apparatuses (hook and loop system) that may be used to secure the blanket to a mammal, in accordance with an embodiment of the present invention.

FIG. 11 depicts a cooling blanket with two stabilizing apparatuses that may be used to stabilize the blanket with respect to a mammal, in accordance with an embodiment of the present invention.

FIG. 12 depicts a case to hold a cooling blanket, in accordance with an embodiment of the present invention.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. March, Advanced Organic Chemistry Reactions, Mechanisms and Structure 4th ed., J. Wiley & Sons (New York, N.Y. 1992) provides one skilled in the art with a general guide to many of the terms used in the present application.

Moreover, one skilled in the art will recognize many methods and materials similar or equivalent to those described herein, which could be used in the practice of the present invention. Indeed, the present invention is in no way limited to the methods and materials described. For purposes of the present invention, the following terms are defined below.

The various embodiments of this invention may be used with any animals for which it would be desirable to effect body cooling, as will be readily appreciated by healthcare and veterinary practitioners. The term “mammals,” as used herein, thus refers to any member of the class Mammalia, including, without limitation, humans and non-human primates such as chimpanzees, and other apes and monkey species; farm animals such as cattle, sheep, pigs, goats and horses; domestic mammals such as dogs and cats; laboratory animals including rodents such as mice, rats and guinea pigs, and the like.

“Reagent,” as used herein, refers to any substance that can react with one or more substances to produce an endothermic reaction. Suitable reagents include, but are not limited to, ammonium nitrate (which may be coated with a conditioning agent to improve handling and storage), urea, and water. The reagent may exist in any convenient state or form for purposes of manufacturing, configuring, transporting or otherwise using the device of the present invention. For example, the reagents may be in a gaseous, liquid, or solid state; they may be formed as crystals, pellets, rods, and/or other chemical or mechanical forms; they may be used alone or in any suitable combination with one another; and they may be used in a salt form, a purified form, and/or combined with other generally recognized additives, ingredients, excipients, preservatives, carriers, or the like.

The present invention is based on a cooling blanket configured to cover at least the torso (i.e., the entire body, excluding the head, neck, limbs, and appendages) of a mammal. In alternate embodiments of the present invention, the cooling blanket may be configured to cover additional parts of the body, such as the head, neck, and/or one or more limbs. In still further embodiments of the present invention, a cooling blanket may be configured to cover the torso of a mammal, while one or more additional blankets may be configured to be interconnected with the cooling blanket to cover not only the torso, but also additional parts of the body, such as the head, neck, and/or one or more limbs.

The cooling blanket may be in any desirable configuration, depending upon its intended purpose and the size and anatomy of the particular mammal that it is designed to cool. For instance, cooling blankets in accordance with present invention that are intended for average-sized human adults will typically be approximately 1-4 ft. in width and approximately 2-5 ft. in length, which is likely to be larger than those designed for human children. Also, cooling blankets for certain mammals may be configured with shapes somewhat different than those illustrated herein (which are designed generally for humans and primates) to account for differential anatomy, such as a cow's utter or a cat's four legs. Similarly, cooling blankets of various sizes may be readily configured for mammals that are substantially above or below the average size, shape, weight, girth, etc. relative to other mammals of the same species. The cooling blanket may be designed to be disposable. All of the aforementioned considerations are contemplated as being well within the scope of the present invention, and corresponding design alterations can be readily implemented by one of skill in the art without undue experimentation to account for the same.

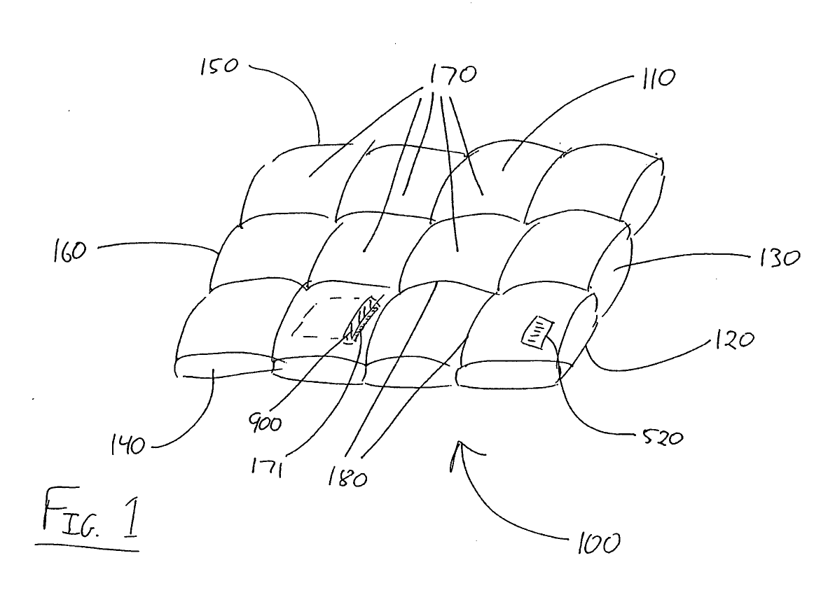

In an embodiment of the present invention, as illustrated in FIG. 1, the cooling blanket 100 may include an outside layer 110, an inside layer 120, a top end 130, side ends 140, 150, and a bottom end 160. The inside layer 120 is configured to be the surface of the cooling blanket 100 that is in closest proximity to and/or in contact with the mammal, while the outside layer 110 is configured to be an external surface. The cooling blanket 100 may be separated into one or more compartments 170 by adjoining layers 110 and 120 along seams 180.

The side ends 140, 150 may be configured as relatively thicker than other segments of the cooling blanket 100. The side ends 140, 150 may be adapted to be placed under the arms of a human, either once the cooling blanket 100 is laid on top of the human and the side ends 140, 150 are wrapped beneath the arms on either side of the human, or by laying the cooling blanket 100 entirely under the human and resting the arms on top of the side ends 140, 150.

The outside layer 110 may include any convenient number of sub-layers. The sub-layers may be constructed from the same or different material. For example, a sub-layer may be constructed from a pliable plastic to provide flexibility and resilience to the cooling blanket 100, while another sub-layer may be constructed from an insulating material to aid in regulation of system temperature when in use. Particularly suitable pliable plastics may include, but are in no way limited to, polyethylene terephthalate, high density polyethylene, polyvinyl chloride, vinyl, low density polyethylene, polypropylene, polystyrene, resins (e.g., surlyn ionomer resins). Multiple sub-layers may be constructed from the same materials; for instance, the outside layer 110 may include multiple sub-layers of one or more types of pliable plastic and/or one or more types of insulating material.

The inside layer 120 may also include any convenient number of sub-layers. The sub-layers can be constructed of the same or different material. For example, a sub-layer may be constructed of a material that is suitable for being placed in direct contact with a mammal (e.g., with the mammal's skin); for example, non-tearing paper or cotton may provide a comfortable surface for the mammal, while sub-layers similar to those noted above with respect to the outside layer 110 may additionally or alternatively be used in the inside layer 120. Multiple sub-layers may be constructed of the same materials; for instance, the inside layer 120 may include multiple sub-layers of one or more types of non-tearing paper or cotton and/or multiple sub-layers of one or more types of pliable plastic and/or one or more types of insulating material.

Each compartment 170 may contain one or more reagents 101, 102 in a variety of different configurations, depending upon the particular reagents 101, 102 used in connection with alternate embodiments of the present invention. In each configuration, the cooling blanket 100 is adapted to be activated (i.e., to generate a cold temperature to cool the mammal) when at least one reagent 101 is placed in contact with at least one other reagent 102; the reagents 101, 102 being pre-selected to produce an endothermic reaction upon contact with one another. A variety of reagent combinations may be used for this purpose, such as, by way of example, ammonium nitrate and water.

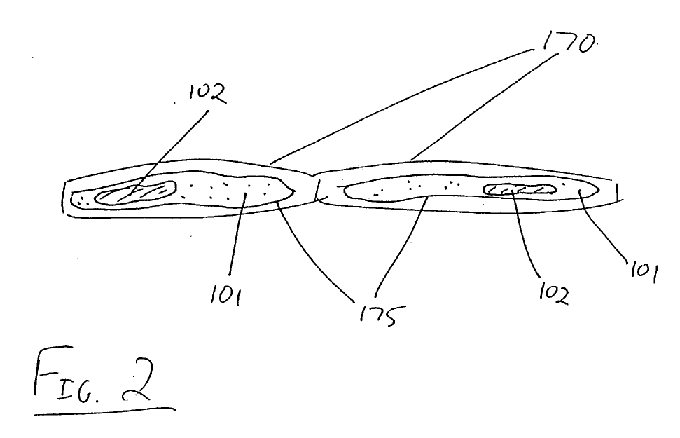

Therefore, in one embodiment of the present invention, as depicted in FIG. 2, each compartment 170 is configured to receive a cold pack 175, such as any suitable cold pack 175 that is commercially available and which can be activated by the application of external pressure (thereby causing two reagents 101, 102 contained therein to mix). The cooling blanket 100 may be manufactured with such cold packs 175 already inside the compartments 170. The compartments 170 may each be configured with an opening 171 through which a cold pack 175 can be inserted and/or removed. This may enable the cooling blanket 100 to be reused, as used cold packs can be removed and new, unused cold packs can be inserted into each compartment 170. In this embodiment, each compartment 170 may be further configured with an external closure 900, such as one or more grommets, one or more tics, one or more buttons, a hook and loop closure, a zipper closure, or any other similar closure means that permits the removable introduction of a cold pack into a compartment 170.

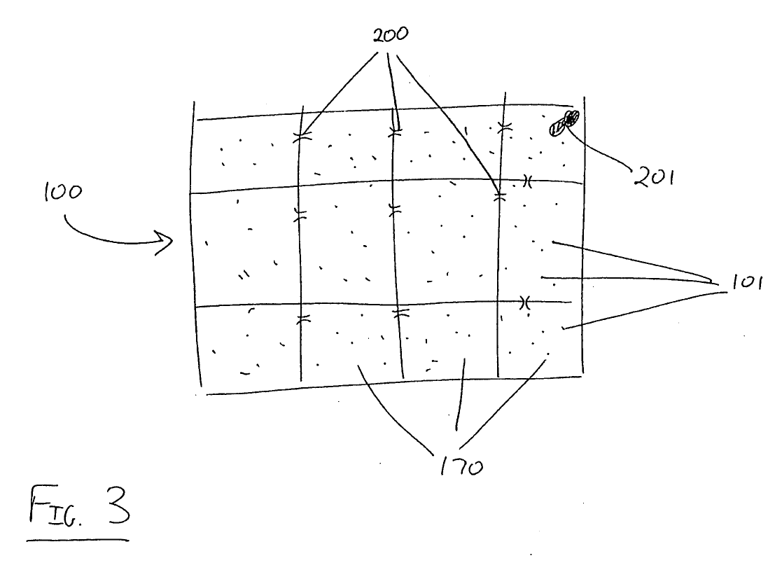

In another embodiment of the present invention, as depicted in FIG. 3, each compartment 170 is configured with at least one one-way valve 200 to create a network that provides one-way, fluid communication through multiple compartments 170 in the cooling blanket 100. In one embodiment, this may provide one-way, fluid communication through all compartments 170 in the cooling blanket 100. In this embodiment, one or more reagents 101 (e.g., granulated ammonium nitrate) is prepackaged in each compartment 170 that has been fitted with a one-way valve 200, while another reagent 102 in fluid form (e.g., water) may be added to the blanket 100 through an inlet valve 201. The fluid reagent 102 may thus flow (in one direction only owing to the one-way nature of the one-way valves 200) through the network of compartments 170 in fluid communication with one another, whereby the fluid reagent 102 contacts the other reagent 101 in each compartment 170. The cooling blanket 100 may thus be activated.

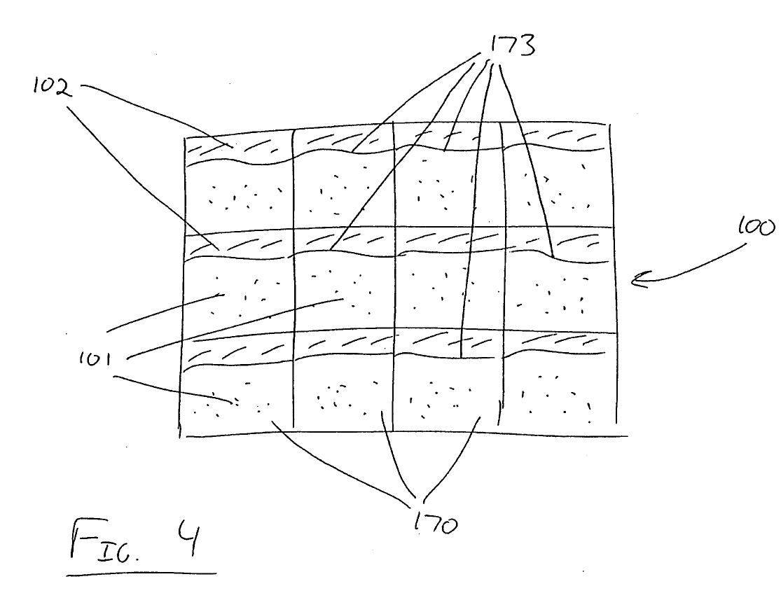

In another embodiment of the present invention, as depicted in FIG. 4, each compartment 170 may be configured with a seal 173 that separates the reagents 101, 102 from one another. Upon application of a predetermined amount of pressure to the compartment 170 by external force, the seal 173 may be configured to rupture, thus allowing the mixing of the two reagents 101, 102 and, thus, the activation of the cooling blanket 100.

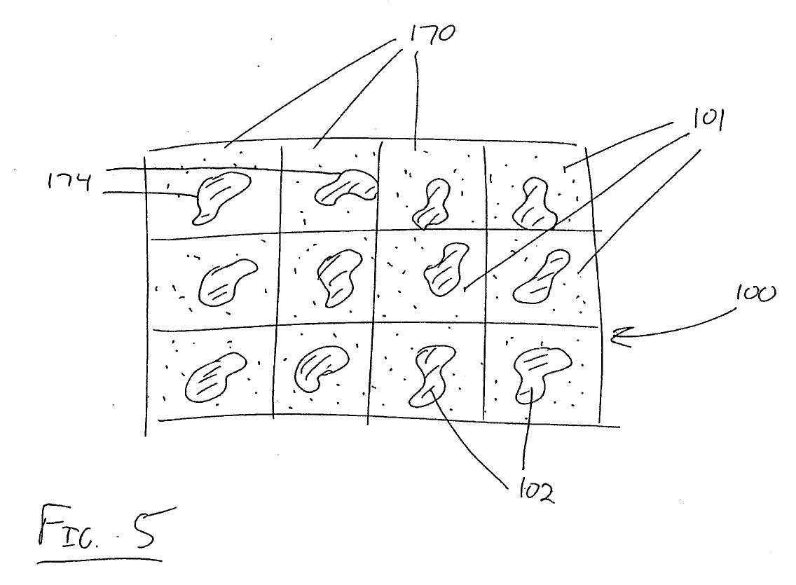

In still another embodiment of the present invention, as depicted in FIG. 5, each compartment 170 may be configured with a sack 174 that includes one or more reagents 102. The sack 174 may be mechanically affixed to an interior surface of the compartment 170, or it may float freely therein. One or more other reagents 101 may be filled in the same compartment 170 outside of the sack 174. Upon application of a predetermined amount of pressure to the compartment 170 by external force, the sack 174 may be configured to rupture, thus allowing the mixing of the reagents 101, 102 and, thus, the activation of the cooling blanket 100.

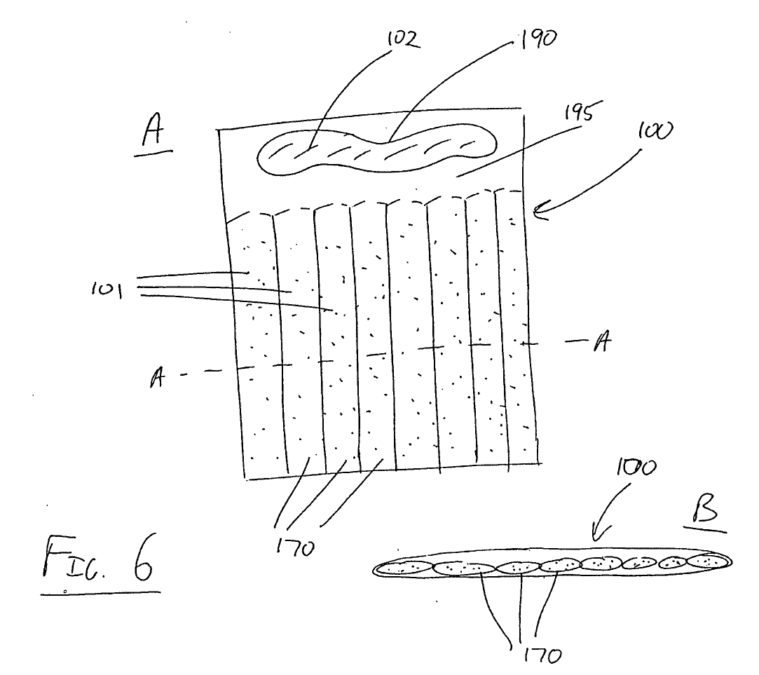

In yet another embodiment of the present invention, as depicted in FIG. 6, the cooling blanket 100 may include a bladder 190, configured to hold a quantity of a fluid reagent 102, which, in one embodiment, may be water. The cooling blanket 100 may also include a series of compartments 170 within which a second reagent 101 is dispersed; the compartments 170 being in fluid communication with the internal portion 195 of the cooling blanket 100 adjacent to the bladder 190. Upon application of a predetermined amount of pressure to the bladder 190 by external force, the bladder 190 may be configured to rupture, thus releasing a quantity of the reagent 102 contained therein to the internal portion 195 of the cooling blanket 100 adjacent to the bladder 190, and thereby allowing the mixing of the reagents 101, 102 in the compartments 170 and, thus, activation of the cooling blanket 100.

A thermometer may be placed on one or more compartments 170, by techniques known in the art, to enable a determination as to whether the compartment 170 has been activated. A detection of a cold temperature may indicate that the compartment 170 is so activated. The thermometer may be, for example, a Tempa-DOT™ thermometry system (commercially available from Minnesota Mining & Mfg. Co. (3M); St. Paul, Minn.).

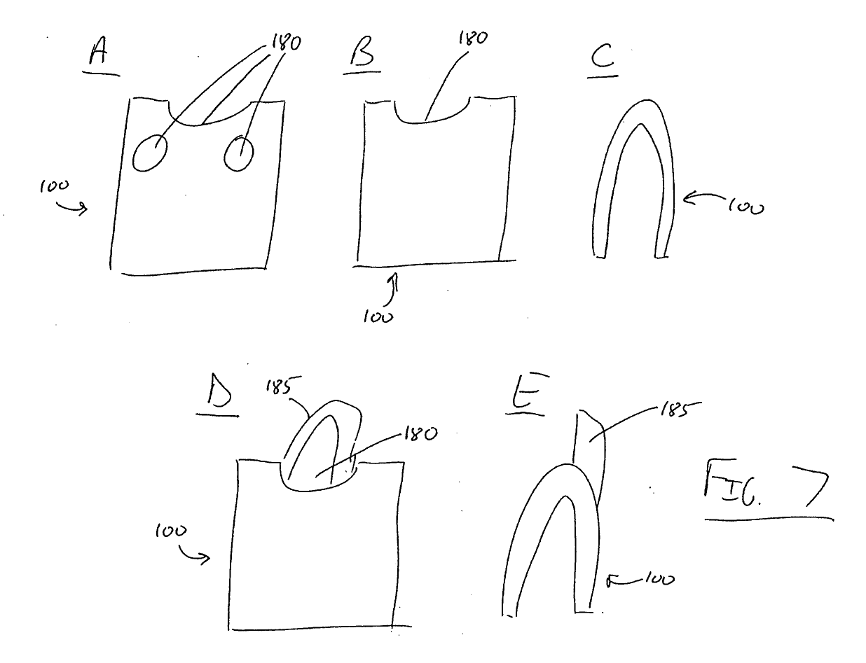

As illustrated in FIG. 7, the cooling blanket 100 may include one or more openings 180. This may be advantageous to allow a mammal's appendages to pass through the cooling blanket 100, and the openings 180, therefore, may be configured at anatomically convenient locations. For instance, where the cooling blanket 100 is configured for human use, two openings 180 may be included to allow the arms to pass through the cooling blanket 100 and/or one opening 180 may be included to allow the head and neck to pass through the cooling blanket 100. In other embodiments (not shown), openings 180 may be included to account for the mammal's other limbs and appendages (e.g., legs, tail, utter). In still further embodiments, cooling blanket 100 may include a hood 185 to at least partially cover a mammal's head. The hood 185 may include one or more compartments 170 to permit the cooling of the mammal's head by techniques similar to those described above.

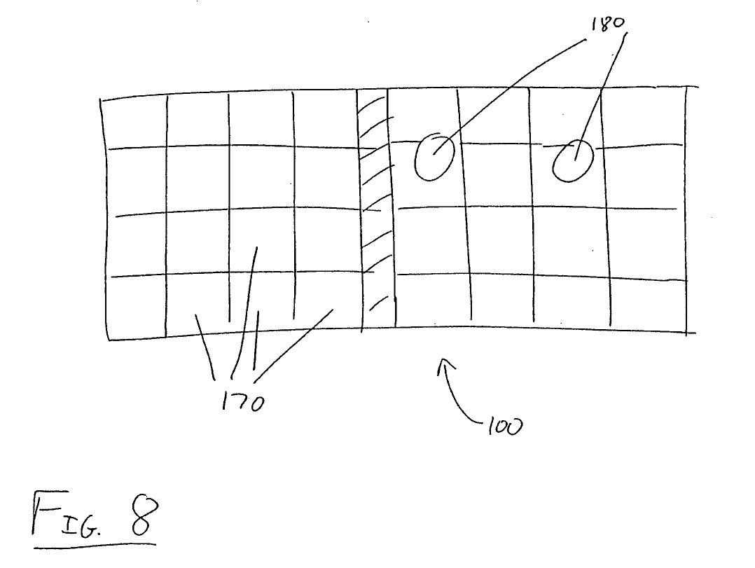

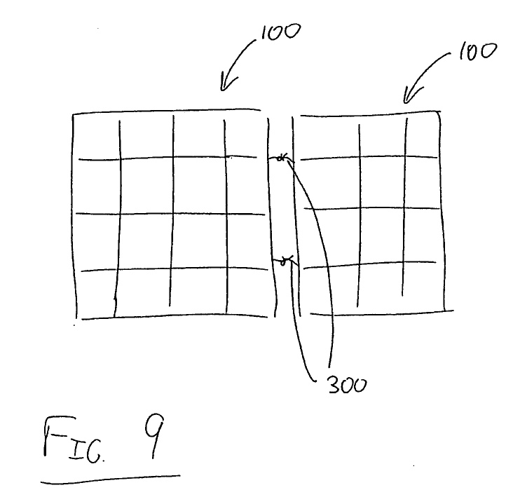

In an alternate embodiment, a cooling blanket system may be configured such that it may be partially or entirely wrapped around a mammal. As depicted in FIG. 8, this can be accomplished by a single, large, unitary cooling blanket. Or, it can be accomplished by constructing a cooling blanket system that includes two or more cooling blankets 100 mechanically secured to one another, as illustrated in FIG. 9. In the latter embodiment, the two or more cooling blankets may be fastened to one another by a fastening apparatus 300.

As also depicted in FIG. 9, fastening apparatus 300 may include a grommet system, a tie system, a button system, a hook and loop system (e.g., a Velcro® hook and loop system), a zipper system, or any other system as will be readily apparent to those of skill in the art and can be used in connection with alternate embodiments of the present invention without undue experimentation. The fastening apparatus 300 may be configured at any convenient location on the cooling blanket 100, such as at its corners and/or at any location along the top end 130, side ends 140, 150, or bottom end 160.

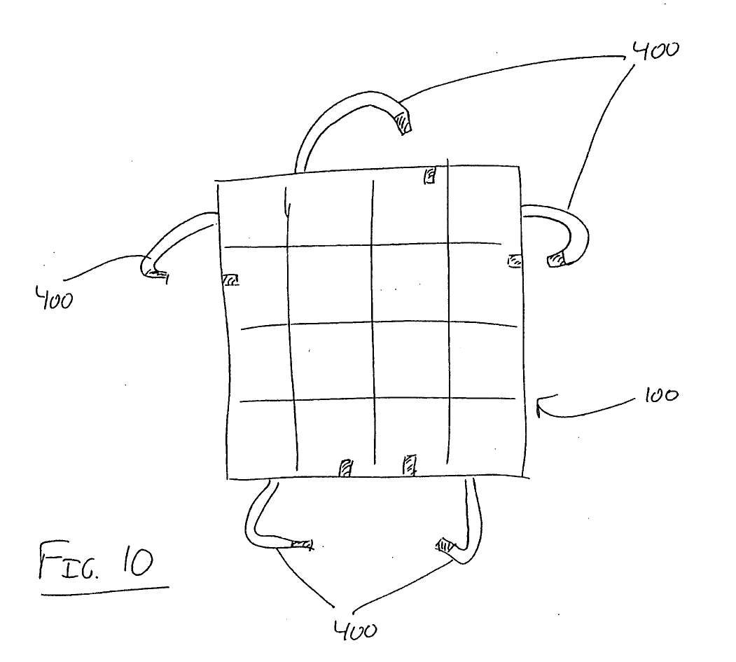

In another embodiment of the present invention, depicted in FIG. 10, a cooling blanket 100 may be secured to the mammal with a securing apparatus 400. Securing apparatus 400 may be used to secure the cooling blanket 100 to the mammal at the neck, arms, legs or another limb or appendage. Securing apparatus 400 may include a grommet system, a tie system, a button system, a hook and loop system (e.g., a Velcro® hook and loop system), a zipper system, or any other system as will be readily apparent to those of skill in the art and can be used in connection with alternate embodiments of the present invention without undue experimentation.

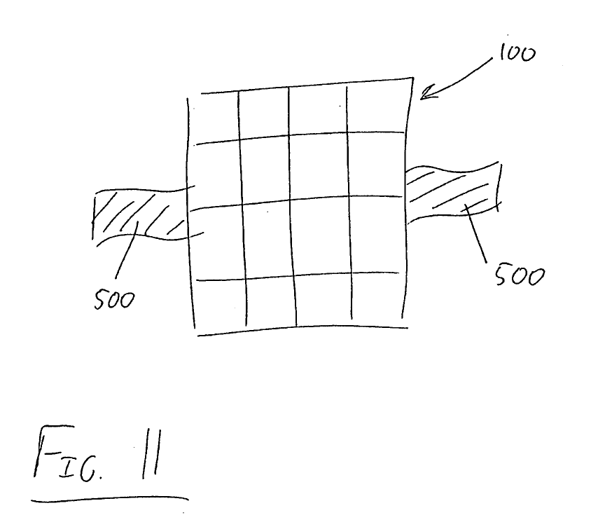

In another embodiment of the present invention, as depicted in FIG. 11, stabilizing apparatus 500 may be included to stabilize the cooling blanket 100 with respect to the mammal, when the mammal is lying prostrate either on its anterior or posterior side. Stabilizing apparatus 500 may include a piece or pieces of material that extend from cooling blanket 100 at one or both side ends 140, 150. In use, stabilizing apparatus 500 may be tucked under the mammal, it may be tucked under the surface on which the mammal is laying (e.g., the stretcher, gurney, bed, floor, etc.), or it may be mechanically affixed to a backboard (not shown).

Various embodiments of the present invention include one or more fastening apparatuses, securing apparatuses, and/or stabilizing apparatuses. These apparatuses need not be used together in any particular combination, and any one can be used by itself in connection with a cooling blanket.

Instructions 520 may be printed on the cooling blanket 100. Examples of instructions 520 that may be especially suitable include, but are not limited to, Advance Cardiac Life Support (“ACLS”) algorithms and drug doses. Types of ACLS algorithms include, but are not limited to, ACLS algorithms for Asystole, Bradycardia, Cardioversion, Pulseless Electrical Activity (“PEA”), Tachycardia, Ventricular Fibrillation/Pulseless Ventricular Tachycardia (“VF/PVT”). This may aid a healthcare practitioner providing care to a patient in the course of a deleterious cardiac episode.

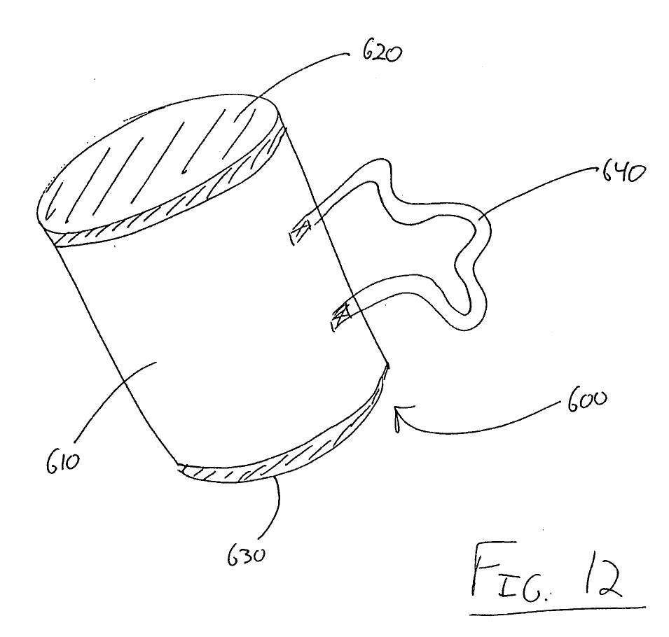

As illustrated in FIG. 12, a case 600 may be used to hold the cooling blanket 100. The case 600 may be configured as a hollow unit 610 with a top 620 and a bottom 630. The case 600 may be configured in any convenient shape, such as cylindrical. The top 620 and/or bottom 630 may be removable. The top 620 and/or bottom 630 may be used in activating the cooling blanket 100 (e.g., using the top 620 and/or bottom 630 to apply the external pressure to compartments 170 of the cooling blanket 100 required to activate it). In one embodiment, the cooling blanket 100 is stored, rolled or folded, inside the case 600. The case 600 may be rigid or soft. In one embodiment, the case 600 is constructed from plastic. The case 600 may have one or more straps 640 that can be wrapped around it to squeeze and activate the cooling blanket 100, by applying a sufficient amount of external pressure. It may thus be removed from the case 600 already activated and ready for immediate use.

The present invention is also directed to a kit to lower the body temperature of a mammal. The kit is an assemblage of materials or components, including at least one cooling blanket. The exact nature of the components configured in the inventive kit depends on its intended purpose. For example, some embodiments are configured for the purpose of treating an adult human. Other embodiments may be configured for treating a child or an infant human. Instructions for use may be included in the kit. “Instructions for use” typically include a tangible expression describing the technique to be employed in using the components of the kit to effect a desired outcome, such as to activate the cooling blanket and/or to lower the body temperature of a mammal. Optionally, the kit also contains other useful components, such as, additional cold packs, containers of water, a water pumping device, ACLS algorithms and drug doses, or other useful paraphernalia as will be readily recognized by those of skill in the art.

The materials or components assembled in the kit can be provided to the practitioner stored in any convenient and suitable fashion that preserves their operability and utility. The components are typically contained in suitable packaging material(s). As employed herein, the phrase “packaging material” refers to one or more physical structures used to house the contents of the kit, such as the cooling blanket. The packaging material is constructed by well known methods. Thus, for example, a package can be a case or bag to contain the cooling blanket. The packaging material generally has an external label which indicates the contents and/or purpose of the kit and/or its components.

### EXAMPLE

The following example is provided to better illustrate the claimed invention and is not to be interpreted as limiting the scope of the invention. To the extent that specific materials are mentioned, it is merely for purposes of illustration and is not intended to limit the invention. One skilled in the art may develop equivalent components without the exercise of inventive capacity and without departing from the scope of the invention.

A cooling blanket is provided with multiple compartments, each of which includes a seal separating a prepackaged quantity of ammonium nitrate from a prepackaged quantity of water. External pressure is applied to the cooling blanket to rupture each of the seals, causing the water and ammonium nitrate to mix, thereby producing an endothermic reaction that creates a cooling effect. The blanket is laid atop a human that is lying prostrate.

While the description above refers to particular embodiments of the present invention, it should be readily apparent to people of ordinary skill in the art that a number of modifications may be made without departing from the spirit thereof. The accompanying claims are intended to cover such modifications as would fall within the true spirit and scope of the invention. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

What is claimed is:

1. A method for treating and/or reducing the likelihood of developing and/or reducing the severity of a condition in a mammal in need thereof, comprising:
providing an apparatus for lowering the body temperature of the mammal, comprising:
a top layer,
a bottom layer, and
at least one compartment formed between the top layer and the bottom layer, the at least one compartment containing a first reagent, and a bladder containing a second reagent;
introducing the second reagent to the first reagent by rupturing the bladder, wherein the first reagent and the second reagent produce an endothermic reaction upon mixing; and
applying the apparatus to the torso of the mammal to treat and/or reduce the likelihood of developing and/or reduce the severity of the condition.

2. The method of claim 1, wherein the condition is selected from the group consisting of head trauma, stroke, heat stroke, hyperthermia, fever, seizure, neonatal asphyxiation, ailments associated with drowning, respiratory failure, cardiac event, acute myocardial infarction, sepsis, and combinations thereof.

3. The method of claim 1, wherein the first reagent is selected from the group consisting of ammonium nitrate, urea, water, and combinations thereof.

4. The method of claim 1, wherein the bladder is ruptured by applying a predetermined amount of external force.

5. The method of claim 1, wherein the apparatus further comprises at least one opening, and the method further comprises extending a limb or an appendage of the mammal through said at least one opening.

6. The method of claim 1, wherein the apparatus further comprises a hood, and the method further comprises covering the head of the mammal with the hood.

7. The method of claim 1, wherein the apparatus further comprises a securing device, and the method further comprises securing the apparatus to the mammal at its neck, limb or appendage with the securing device.

8. The method of claim 1, further comprising monitoring the body temperature of the mammal.

9. A method for treating and/or reducing the likelihood of developing and/or reducing the severity of a condition in a mammal in need thereof, comprising:
providing an apparatus for lowering the body temperature of the mammal, comprising:
a top layer,
a bottom layer, and
at least one compartment formed between the top layer and the bottom layer, the at least one compartment containing a first reagent, and an inlet valve adapted to permit the introduction of a second reagent;
introducing the second reagent to the first reagent through the inlet valve, wherein the first reagent and the second reagent produce an endothermic reaction upon mixing; and
applying the apparatus to the torso of the mammal to treat and/or reduce the likelihood of developing and/or reduce the severity of the condition.

10. The method of claim 9, wherein the condition is selected from the group consisting of head trauma, stroke, heat stroke, hyperthermia, fever, seizure, neonatal asphyxiation, ailments associated with drowning, respiratory failure, cardiac event, acute myocardial infarction, sepsis, and combinations thereof.

11. The method of claim 9, wherein the first reagent is selected from the group consisting of ammonium nitrate, urea, water, and combinations thereof.

12. The method of claim 9, wherein the apparatus further comprises at least one opening, and the method further comprises extending a limb or an appendage of the mammal through said at least one opening.

13. The method of claim 9, wherein the apparatus further comprises a hood, and the method further comprises covering the head of the mammal with the hood.

14. The method of claim 9, wherein the apparatus further comprises a securing device, and the method further comprises securing the apparatus to the mammal at its neck, limb or appendage with the securing device.

15. The method of claim 9, further comprising monitoring the body temperature of the mammal.

16. A method for treating and/or reducing the likelihood of developing and/or reducing the severity of a condition in a mammal in need thereof, comprising:
providing an apparatus for lowering the body temperature of the mammal, comprising:
a top layer,
a bottom layer,
at least one compartment formed between the top layer and the bottom layer, the at least one compartment containing a first reagent, and
a bladder in fluid communication with the at least one compartment, the bladder containing a second reagent and a rupturable seal separating the second reagent from the at least one compartment;
introducing the second reagent to the first reagent by severing the rupturable seal, wherein the first reagent and the second reagent produce an endothermic reaction upon mixing; and
applying the apparatus to the torso of the mammal to treat and/or reduce the likelihood of developing and/or reduce the severity of the condition.

17. The method of claim 16, wherein the first reagent is selected from the group consisting of ammonium nitrate, urea, water, and combinations thereof.

18. The method of claim 16, wherein the condition is selected from the group consisting of head trauma, stroke, heat stroke, hyperthermia, fever, seizure, neonatal asphyxiation, ailments associated with drowning, respiratory failure, cardiac event, acute myocardial infarction, sepsis, and combinations thereof.

19. The method of claim 16, wherein the apparatus further comprises at least one opening, and the method further comprises extending a limb or an appendage of the mammal through said at least one opening.

20. The method of claim 16, wherein the apparatus further comprises a hood, and the method further comprises covering the head of the mammal with the hood.

21. The method of claim 16, wherein the apparatus further comprises a securing device, and the method further comprises securing the apparatus to the mammal at its neck, limb or appendage with the securing device.

22. The method of claim 16, further comprising monitoring the body temperature of the mammal.
